(19) United States
(12) Patent Application Publication
(10) Pub. No.: US 2009/0124927 A1
(43) Pub. Date: May 14, 2009

(54) Endoscopic system for lung biopsy and biopsy method of insufflating gas to collapse a lung

(75) Inventors: Roy Sing-Fatt Chin, Pleasanton, CA (US); Murali Dharan, Danville, CA (US)

Correspondence Address: Katherine N. Addison Esq., 1930 Whitley Avenue, Apt 104, Hollywood, CA, 90068, US

(73) Assignee: Chest Innovations, Inc., Fremont, CA (US)

(21) Appl. No.: 12/269,884

(22) Filed: Nov. 13, 2008

Related U.S. Application Data
(60) Provisional application No. 61/002,953, filed on Nov. 13, 2007.
(60) Provisional application No. 61/067,526, filed on Feb. 28, 2008.
(60) Provisional application No. 61/073,971, filed on Jun. 19, 2008.

(51) Int. Cl. A61B 10/02; A61B 1/018

(52) U.S. Cl. 600/562; 600/106

(57) ABSTRACT

An endoscopic biopsy system comprising a means for drawing a sample to be sealed, separated, and/or collected towards an instrument, such as a structure that includes one or more of: an extendable wire, an extendable mast, an extruded tube with at least one opening and a vacuum therein, a pivot point, a hinge, and a mounting block. The system should have a means to transfer energy to a sample after a sample is grasped, such as a conducting wire or an anvil. The system should also have an airtight means to remove a separated sample from a biopsy site for analysis, such as a collection bag or an internal vacuum suction system. The endoscopic biopsy system can be used in a method of obtaining a biopsy from the thoracic cavity that includes a step of insufflating gas to induce pneumothorax and collapse a lung.

### BACKGROUND OF THE INVENTION

1. Field of the Invention

The present invention relates to an endoscopic biopsy system comprising an instrument that can seal surrounding tissue(s), separate sample(s), and/or collect sample(s) from the interior of a subject for removal and analysis and to a method of obtaining biopsy samples using the system in accordance with minimally invasive surgical techniques.

2. Description of the Related Art

Several systems, devices, and methods have been proposed for obtaining biopsy samples. Recent developments in the art have focused on the successive collection and removal of multiple samples during one procedure without reinserting the biopsy instrument into the body between samples. U.S. Patent Application Publication No. 2007/0213632 (Okazaki et al.) and U.S. Pat. Appl. Pub. No. 2006/0258955 (Hoffman et al.) disclose such systems.

Another focus area for recent developments has been on methods used to separate tissue from its surrounding attachments and seal or secure the area to reduce drainage. Historically, mechanical cutting techniques were used to severe tissue with sharp instruments. The sites from which samples were removed were repaired or sealed together using staples or sutures. Another visit to the doctor was often required for removal of the staples or sutures after sufficient wound healing and new tissue growth had occurred. Modern techniques aim to seal off an area from which a sample is to be cut prior to or contemporaneously with the cutting in order to reduce drainage and bleeding following cutting. Another characteristic of modern techniques is that they do not use mechanical energy to “cut” in the conventional sense but instead rely upon other forms of energy such as electricity or heat to “cauterize”. For example, U.S. Pat. No. 6,533,778 (Herzon) discloses a thermal cauterizing forceps device with ceramic heater elements mounted within the tips of the forceps' tines. U.S. Pat. Appl. Pub. No. 2006/0217706 (Lau et al.) also discloses an apparatus that relies upon thermal energy, generated by the electrical resistance of various heating elements coupled with the electricity passing through those elements, to weld and cut tissue. The structure of the apparatus disclosed in Lau et al. is relatively straight-forward, utilizing two elongated jaws, analogous to conventional mechanical cutting with scissors.

Regardless of the form of energy (i.e. cryogenic, electrical, mechanical, microwave, laser, thermal, ultrasound, etc.) used to seal and/or separate tissue, the cutting elements of the biopsy instruments through which that energy is applied are amenable to a myriad of structural configurations. For example, U.S. Pat. Appl. Pub. No. 2007/0213633 (McClellan) discloses an angled (rather than blunt) coring needle having sharp edges, a cross-section with one flat side and another converging side, and a flexible shearing door situated therein to separate a sample. The problem with this device is that it would take a very long time to collect a superficial layer of tissue from a large surface area. U.S. Pat. Appl. Pub. No. 2007/0213634 (Teague) discloses a plurality of barbs for cutting. The barbs may have various configurations (convex extending distally parallel to the longitudinal direction of the sampling device, convex extending proximally parallel, and convex extending perpendicular) arranged throughout the distal end of an elongated member. The problem with this device is that the sharp, scattered barbs are not moveable and are difficult to control precisely for grasping a particular area of tissue while leaving its vicinity unharmed. Further, the barbs are likely to shred the sample into small fragments as it is collected (in a manner similar to a cheese or vegetable grater) which can destroy certain characteristics of the tissue desired for analysis. Thus, the barb shredder system does not appear to be conducive to the collection of large, intact volumes of specimens. In contrast, some embodiments of the present invention are capable of obtaining intact specimens of 3 mm diameter by 10 mm length.

One aspect of the present invention is to set forth more efficient structures for the sealing, separation, and collection of biopsy samples. The structures of the present invention make use of extendable wires (stiff and flexible), pivot points, anvils, slideably mounted elements, and extruded tubes. These elements facilitate the separation of samples from a greater variety of surfaces than conventional forceps, scissor-jaws, and coring biopsy needles. The present invention also provides elements for biopsy collection such as vacuum suction ports, a mesh-netting network, and expandable/retractable collection bags in the immediate vicinity of the separation site. The following embodiments have been designed for biopsy sample site sealing, separation, and/or collection: (i) the “spring load wires” embodiment, (ii) the “mast and spinnaker” embodiment, (iii) the “lasso” embodiment, (iv) the “octo-arm”/“octo-mat” embodiment, and (v) the “stiff mast with tissue grab and capture technology” embodiment.

Another aspect of the present invention is to teach biopsy instruments suited for sampling along planar surfaces, such as the wall of a lung. Unlike nodular or pedunculated sample sites having tissue easily grasped with forceps, planar surfaces are traditionally more difficult to biopsy without destroying the sample or significantly damaging nearby healthy tissue. The devices and methods described herein, such as the “stiff mast with tissue grab and capture technology” embodiment and its method of use, permit the sampling of sensitive planar regions without shredding the specimen or cutting too deeply.

With respect to biopsy systems, devices, and methods designed for the pleural region in particular, recent efforts have been directed towards minimally invasive techniques using smaller incisions and local anesthesia For example, see “Textbook of Pleural Diseases” by Richard W. Light and Y. C. Gary Lee (London: 2003, Edward Arnold Publishers, Ltd.), especially chapters 39-41. Larger incisions with conventional cutting methods generally produce substantial drainage that must be addressed post-surgery by the insertion of a chest tube. If a chest tube is nonetheless necessary despite the use of modern procedures, smaller tube sizes are generally less painful and may be adequate if the amount of fluid and other materials to drain can be sufficiently reduced by improved sealing methods. If fluids and other materials need to be drained post-surgery, the insertion of a chest tube is important to prevent pneumothorax. Pneumothorax is the accumulation of gas, such as air, in the pleural cavity between the visceral pleura lining the lungs and the parietal pleura lining the chest wall. Conventional rigid chest tubes are frequently painful and conventional flexible chest tubes frequently kink or buckle requiring painful manipulation or reinsertion.

One objective of this invention is to reduce or eliminate the need for chest tubes by eliminating fluids and materials to be drained through small incisions, narrow samples, rapid sealing near the time of separation, and efficient on-the-spot material collection techniques, such as vacuum suction devices, with a large surface area relative to the incision site. In the event a chest tube is required, one aspect of this invention is to disclose a novel painless, kink-less, non-buckling chest tube that is easy to insert and remove and provides rapid drainage.

### BRIEF SUMMARY OF THE INVENTION

An endoscopic biopsy system of the present invention comprises, an instrument capable of sealing, separating and/or collecting biopsy samples and designed for insertion through a catheter or the working channel of an endoscopic instrument including a Chest Innovations (trademark) minithoracoscope (trademark). The instrument of the present invention is designed for insertion via a small incision site such as those made using minimally invasive surgical techniques. In some of its embodiments, the endoscopic biopsy system of the present invention is designed to acquire biopsy samples that are sufficiently narrow (i.e. 1-4 mm diameter) to fit through the small working channels of endoscopic instruments (i.e. cannulas, catheters, trocars, thoracoscopes, etc.). However, the samples may be much longer than their width (i.e. having lengths of 10 mm or 2-10 times the diameter width) in order that a sample size of sufficient volume for analysis is acquired.

The sealing, separating and collecting functions of the endoscopic biopsy system of the present invention occur internally within the patient from which the sample is taken. Therefore, there is no exposure of separated biopsy sample materials (i.e. blood, fluid, cells, tissue, DNA, RNA, etc.) to the external environment prior to the time they are ready for analysis, thereby avoiding the risks of sample contamination and degradation. The collection methods and devices of the present invention are sealed and airtight. These include the use of a collection bag and the use of a tubular vacuum suction system that draws separated samples through internal channels to a sealed external collection chamber.

According to one aspect of the present invention, the endoscopic biopsy system comprises a device with spring loaded wires. In this embodiment two or more long, stiff wires protrude from an instrument and are joined together at their distal tips. A pivot point with a range of angular motion is positioned upon each wire, between the point where the wires leave the instrument and their distal tips. When the device is in its open position for grasping a sample, the angle of the pivot point is less than 180 degrees, approaching 90 degrees. When the device is in its closed position for sealing and separating a sample held between the wires, the angle of the pivot point should be greater than 90 degrees, approaching 180 degrees. An anvil is positioned along each wire on the distal segment of wire between the pivot point and the distal tip of the wire. At least one anvil is used to transmit energy to seal and separate a biopsy sample from its surrounding environment. At least one anvil is used as a buttress to hold the sample in position, creating pressure to seal and severe when operating in association with another anvil supplying energy. The anvil that transmits energy may also serve as a buttress. A collection bag is attached to the wires so that separated tissue moves into the collection bag which is then sealed and removed from the body.

According to another aspect of the present invention, the endoscopic biopsy system comprises a rigid mast extendable through an instrument. One or more wire extends from an opening within the tubular rigid mast and joins to the mast at its distal tip. At least one wire is capable of transmitting energy to a site to be biopsied in order to seal surrounding bodily connections and separate a sample. At least one wire has a collection bag secured thereto for receiving a separated sample and securely removing the sample from the body. In its open position for grasping a sample, the wire extends from the longitudinal base of the mast in the shape of a rainbow or an arch. Once a sample enters the arch and is positioned between a sealing and separating, energy-transmitting wire on one side, and the rigid mast on the other side, the wire is retracted and drawn parallel along the body of the mast. The mast provides a buttress against which the energy-transmitting wire comes into contact with the sample, creating pressure to transfer energy to the sample sufficient to seal and/or separate it.

According to another aspect of the present invention, the endoscopic biopsy system comprises two or more wires extendable from an instrument and attached at their distal tips with anvils slideably mounted thereon. Each wire has at least one anvil slideably mounted thereon. At least one anvil comprises an energy source element for sealing and/or separating tissue. Anvils without energy source elements are used as buttresses for the anvils with the energy source elements to press against as they come into contact with tissue to create pressure and transfer energy. In their open position for grasping a sample, the wires repel from one another to create a space between them for receiving tissue to be sealed and separated. In their closed position for transferring energy from the energy source on an anvil to the tissue, the wires are attracted to one another as they become parallel. In the closed position, an anvil with an energy source element thereon is aligned to oppose at least one other anvil (with or without its own energy source element) acting as a buttress. A collection bag may also be attached along the lengths of any two wires in a manner that does not interfere with the sliding movement of the anvils as the bag receives and retains separated tissue.

According to another aspect of the present invention, the endoscopic biopsy system comprises a soft extruded tube (an “octo-arm”) extendable from an instrument, with openings thereupon, with one or more wire therein, and through which vacuum suction may be applied. When a vacuum is applied within the extruded tube, the openings attach themselves to a nearby organ or nearby tissue (i.e. a lung wall). If the instrument is in a cavity, such as the pleural cavity, the openings may also draw in any freely flowing or loosely attached materials within their proximity. The suction draws materials from the biopsy site through airtight sealed channels to a proximal site for removal and analysis. The internal wires of the tube may be used for a variety of purposes including as an observation medium (part of an optical system with direct visualization and/or electronic projection to an external monitor), as a light source (for use with an optical system), or as an energy transfer medium (for sealing and separating suctioned materials).

According to another aspect of the present invention, the endoscopic biopsy system comprises several soft extruded tubes (“octo-arms”), as described in the previous paragraph, joined together by mesh-netting in between adjacent tubes and with a drawstring surrounding their periphery. In this system of tubes (an “octo-mat”), each tube with openings thereupon and one or more wire therein functions the same as it would independently, as described in the previous paragraph. The advantage of this system of tubes is that it has the capacity to expand (via the flexible mesh-netting joints and relaxation of the peripheral drawstring) to cover a larger surface area within a shorter period of time. Further, the network of mesh-netting holding the tubes together secures retrieval of any material missed by the suction system on the first pass. The network of mesh-netting is particularly beneficial when the vacuum pressure is low (i.e. due to equipment constraints or sensitivity at the biopsy site). The network of mesh-netting is also particularly beneficial for use in biopsy sites that are dense with potential sample material because even a high pressure vacuum system is unlikely to be able to keep pace with the rate at which the tubular openings come into contact with sample material. The mesh-netting network affords the vacuum system a second, third, etc. chance to capture material as it rebounds from the netting to approach the tubular openings.

According to another aspect of the present invention, the endoscopic biopsy system comprises one or more stiff extruded tube and one or more flexible extruded tube attached to a support wire by flexible hinges and blocks slideably mounted along the wire. Each tube has openings thereupon and vacuum suction may be applied through the interior of each tube. Also provided is at least one energy-transfer wire for sealing the surrounding environment of a biopsy sample and separating the sample from that environment. A stiff tubular mast extends from an instrument to support the wires and each wire extends form an opening within the mast. Optionally, a collection bag may be provided which is mounted upon the stiff mast on one side and on the other side is either mounted upon the tube support wire or an additional wire. If the collection bag is mounted upon the tube support wire, it should be aligned so as not to interfere with the slideable movement of the blocks through which the tubes are attached.

The endoscopic biopsy system and method of the present invention may be adapted for use with a conventional, reusable endoscope or a disposable, single-use endoscope such as disclosed in U.S. Pat. Appl. Pub. No. 2005/0075538 (Banik et al.) and continuation-in-part U.S. Pat. Appl. Pub. No. 2005/0197536 (Banik et al.).

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a cross sectional view of a catheter through which the instrument of the invention may be inserted. The catheter comprises various channels.

FIG. 2A is a side view of an endoscopic system for lung biopsy according to a first embodiment of the invention. The “spring load wires” embodiment of the sealing and separating instrument is shown extending through the catheter with its pivots and collection bag in the open position for receiving a biopsy sample.

FIG. 2B is another side view of the “spring load wires” (first) embodiment of the invention showing the instrument in a closing position for securing a biopsy sample that has been grasped in order to seal and separate.

FIG. 2C is a view along line A-A′ of FIG. 2B showing a separated biopsy sample secure within the collection bag for removal to a proximal end of the catheter system.

FIG. 2D is a cross sectional view along line B-B′ of FIG. 2C, looking toward the proximal end of the system, showing the electrical wiring channel within the instrument channel.

FIG. 3A is a side view of an endoscopic system for lung biopsy according to a second embodiment of the invention. The “mast and spinnaker” embodiment of the sealing and separating instrument is shown extending through the catheter with its rigid tubular mast, flexible sealing and separating wire, support wire and collection bag in the open position for receiving a biopsy sample.

FIG. 3B is a cross sectional view of the “mast and spinnaker” (second) embodiment of the invention of FIG. 3A.

FIG. 3C is another side view of the “mast and spinnaker” (second) embodiment of the invention showing the wires and collection bag in the closed position for securing a biopsy sample that has been obtained.

FIG. 3D is a cross sectional view of the “mast and spinnaker” (second) embodiment in the closed position along the line C-C′ as shown in FIG. 3C, showing the rigid tubular mast, the sealing and separating wire, the collection bag support wire, the collection bag, and the attachment of the collection bag to the mast.

FIG. 4A is a side view of an endoscopic system for lung biopsy according to a third embodiment of the invention. The “lasso” embodiment of the sealing and separating instrument is shown extending through the catheter with its support wires, sliding anvils, and collection bag in the open position for receiving a biopsy sample.

FIG. 4B is another side view of the “lasso” (third) embodiment of the instrument showing the device in operation with the catheter being advanced as the support wires are retracted and the anvils move along the support wires.

FIG. 4C is another side view of the “lasso” (third) embodiment of the instrument showing the device in its closed position to seal, separate, and capture a biopsy sample between adjacent anvils. The support wires are retracted and the collection bag is expanded and securely sealed.

FIG. 5A is a side view of an endoscopic system for lung biopsy according to a fourth embodiment of the invention. The “octo-arm” embodiment comprises a tube with openings capable of producing a vacuum and an embedded wire for sealing and separating.

FIG. 5B is a cross sectional view of the “octo-arm” of FIG. 5A showing the openings on the top of the tube and the wire embedded in the bottom of the tube.

FIG. 5C is a top view of the “octo-arm” (fourth) embodiment of FIG. 5A showing the openings on the tube through which vacuum suction may be applied.

FIG. 5D is a top view of several interconnected “octo-arms” joined together to form an “octo-mat” by netting in between the arms and a drawstring (or retractable wires) attached to the peripheral arms of the mat.

FIG. 6A is a side view of an endoscopic system for lung biopsy according to a fifth embodiment of the invention. The “stiff mast with tissue grab and capture technology” embodiment of the sealing and separating instrument is shown extending through the catheter with its tubular capture assembly, support wire, and sealing and separating wire in the open position for receiving a biopsy sample.

FIG. 6B is a cross sectional view of the “stiff mast with tissue grab and capture technology” (fifth) embodiment of FIG. 6A.

FIG. 6C is a top view of the “stiff mast with tissue grab and capture technology” (fifth) embodiment of FIG. 6A illustrating an optional collection bag that may be attached to the capture assembly for the collection of a biopsy sample.

FIG. 6D is another side view of the “stiff mast with tissue grab and capture technology” (fifth) embodiment showing the device in a closing position with the support wire of the capture assembly and the sealing and separating wire being retracted toward the stiff mast.

FIG. 6E is a cross sectional view of the “stiff mast with tissue grab and capture technology” (fifth) embodiment as in FIG. 6D showing how the arms of the capture assembly project radially outward in a direction perpendicular to the longitudinal orientation of the stiff mast when the device is in the closing position.

FIG. 6F is a top view of the “stiff mast with tissue grab and capture technology” (fifth) embodiment, as in FIG. 6D, showing how the arms of the capture assembly project radially outward in a direction perpendicular to the longitudinal orientation of the stiff mast when the device is in the closing position.

FIG. 6G is another side view of the “stiff mast with tissue grab and capture technology” (fifth) embodiment showing the device in a closed position with the support wire of the capture assembly and the sealing and separating wire retracted against the stiff mast.

FIG. 6H is a cross sectional view of the “stiff mast with tissue grab and capture technology” (fifth) embodiment, as in FIG. 6G, showing how the arms of the capture assembly align in the same plane and project radially outward in a direction perpendicular to the longitudinal orientation of the stiff mast when the device is in the closed position.

FIG. 6I is a top view of the “stiff mast with tissue grab and capture technology” (fifth) embodiment, as in FIG. 6G, showing how the arms of the capture assembly align in the same plane and project radially outward in a direction perpendicular to the longitudinal orientation of the stiff mast when the device is in the closed position. Also shown is a flexible tubular arm placed at the distal end of the instrument relative to the other stiff tubular arms.

FIG. 6J is a top view of the “stiff mast with tissue grab and capture technology” (fifth) embodiment showing how the flexible tubular arm, positioned distally along the mast relative to the other stiff tubular arms, changes shape during the retraction of the mast and removal of a biopsy sample.

FIG. 6K is a detailed side view of the “stiff mast with tissue grab and capture technology” (fifth) embodiment showing the interrelationship of the various parts of the embodiment including: stiff mast with proximal and distal ends, support wire actuated by a proximal pull, stiff tubular arms positioned proximal to a flexible tubular arm, flexible hinge points on the stiff tubular arms and sliding blocks attached to each tubular arm that move along the support wire.

### DETAILED DESCRIPTION OF THE INVENTION

First, a general procedure for the collection of biopsy samples from a lung according to the systems and methods of this invention will be outlined. Second, various embodiments of the endoscopic biopsy system and various methods for their use will be described in detail with reference to the several drawings. Although the systems and methods of this invention are illustrated with respect to collecting biopsy samples from a lung, the systems and methods are not limited to lung biopsy. One having ordinary skill in the art will recognize that the systems and methods described herein are readily adapted for the collection of biopsy samples from several regions of the body.

### General Procedure

Step One: Consent, Anesthesia, Medical Staff, and Set-Up

Prior to beginning the procedure, the informed consent of the patient should be obtained.

One advantage of the present invention, as compared to traditional open-surgery biopsy techniques, is that it is done under local anesthesia rather than general anesthesia. Consequently, there is less interference with the homeostasis of bodily functions and recovery time is reduced permitting patients to avoid lengthy and expensive post-operative stays in the hospital recovery unit. Further, local anesthesia generally allows for a quicker post-operative assessment of the patient's condition and of the success of the procedure. The preferred drug of choice for local anesthesia in the present procedure is a long-acting local anesthetic agent like bupivacaine. Lidocaine, novacaine, ropivacaine and procaine may also be used. Intravenous sedatives including versed, morphine, fentanyl and other agents enhance the effects of the local anesthetic agent by causing the patient to become sleepier, less anxious, and number to sensations like pain. An anesthesiologist or anesthetist should be required to standby during the biopsy procedure until the operating physician is very comfortable in using the devices described herein.

This procedure is to be done in a procedure room, operative room, or in the ICU (Intensive Care Unit). A RN (Registered Nurse) should be positioned bedside throughout the procedure and sterile precautions should be used. A telemetry unit should be used to monitor heart rate and blood pressure as needed. Oxygen saturation should also be measured throughout the procedure.

Typical endoscopes provide channels for gas and fluid exchange between the external environment and the internal biopsy site. Carbon dioxide or an equivalent gas may be insufflated to the biopsy site through such a channel, during the biopsy procedure, at flow rates of 2-4 liters per minute. Carbon dioxide gas is preferable because it is non-combustible (unlike oxygen), dissolves in blood, and does not cause clots or bubbles when introduced into the rib-restricted thoracic cavity (unlike air). Any other gas having these same advantageous characteristics that is otherwise medically compliant and safe for introduction within the interior of the thoracic cavity may also be used.

The patient's diagnostic data is to be reviewed by a pulmonologist. It is preferable to have CXR (Chest X-Ray) and CI (Computed Tomography) scans readily available. Preferably, a thoracic surgeon on standby should be available for back-up support and assistance.

Step Two: Incision, Insertion of Minithoracoscope, and Insufflation to Induce Pneumothorax

The point of entry is based on the diagnostic data as determined by the pulmonologist. Once the point of entry is determined, the operative site surrounding the point of entry is prepared and draped in a sterile manner.

Next, the local anesthetic agent is infiltrated. A total of 5 mL is usually adequate to anesthetize from the skin to the pleura. A needle is inserted into the intrapleural space. An ease in injection is noted as the needle tip enters the pleural space. This can be confirmed by aspirating air.

A blade knife (size: 11-gauge) is used to make an incision (approximately 2 mm). This incision will facilitate the entry of the Chest Innovations (trademark) (hereinafter, CI) minithoracoscope (trademark). The entry point is always superior to the rib to prevent injury to the intercostal vessels. The CI minithoracoscope has a multi-port minitrocar (trademark) that is held in the midportion of the scope for better directional control. Steady forward pressure is needed to enter the pleural space. Insufflating the internal region during the introduction of the minithoracoscope (or other instruments) is preferred to reduce the possibility of lung injury. Providing continuous insufflation to the internal region of the site to be biopsied also facilitates visualization and prevents fogging of the CI minithoracoscope.

As the pleural space is entered, there is a “give” or sudden drop in pressure, at which time the multi-port minitrocar is removed. Carbon dioxide insufflation continues into the intrapleural space at 2 liters per minute following the removal of the multi-port minitrocar to induce a pneumothorax causing the lung to collapse. When the lung is collapsed, it is easier to visualize, grasp, and manipulate for obtaining a biopsy. It is also easier to reach a greater number of target locations for sampling from a single incision site when the lung is collapsed. During the procedure the intrapleural pressure is maintained at less than 8 mmHg. The anesthesiologist or anesthetist keeps a watch over the blood pressure as excessive carbon dioxide insufflation may cause hypotension, such as from a mediastinal shift as pressure changes in the thoracic cavity push the heart over. In the event of hypotension, the situation can easily be corrected by stopping the flow of carbon dioxide and aspirating the port. Accordingly, it is important to use a low flow rate of carbon dioxide throughout the procedure to avoid rapid fluctuations in blood pressure and intrapleural pressure.

Step Three: Insertion of Camera and Instruments

As an alternative to relying solely upon the tactile sensation of a pressure drop to determine when the pleural space has been entered, a second option is to introduce a CI minithoracoscope with a camera in one of its ports so that insertion of the biopsy needle and insufflation of carbon dioxide are under direct vision. Using this option, the CI minicamera (trademark) is inserted through a port of the minithoracoscope. The location of the CI minithoracoscope within the interior of a patient can be confirmed by visual inspection of the external monitor which receives image signals transmitted by the minicamera. The monitor is usually available with most scope towers. The CI minicamera may need to be defogged occasionally throughout the procedure. Outside of the body, a solution such as “Fred” by Dexide, Inc. or “Dr. Fog” by O.R. Concepts, Inc. (see also U.S. Pat. No. 5,382,297 assigned to Merocel Corporation) can be used to defog the minicamera. Inside of the body, directing the source of carbon dioxide insufflation at the lens of the minicamera may assist to defog.

As the minithoracoscope advances internally through the prospective biopsy region, the pathology is identified and reviewed. Pictures are taken by the minicamera for documentation and correlation with biopsy samples.

Once a target biopsy region is identified based on the images transmitted by the minicamera, the working miniport (trademark) of the minithoracoscope is ready to be used. The miniport is an instrument channel or a fluid/gas exchange channel. The CI mininstruments (trademark), including forceps, staplers, and energy-transferring sealing and separating devices are inserted to obtain biopsy specimens. The specimens are then removed for pathology analysis and/or for culture and sensitivity studies. If bleeding is encountered during the internal manipulation of CI mininstruments, CI minicoagulators (trademark) can be used to promptly control bleeding. Further, CI suction devices are available for aspiration of pleural fluid. Other solutions can also be provided through one of the working miniports of the minithoracoscope and suctioned out after they are utilized. For example, a saline irrigation solution can be introduced to prevent clots. Electrolytic solutions, cooling fluids, cryogenic fluids, chemotherapeutic agents, medicaments, gene therapy agents, contrast agents, and infusion media may also be used. (See U.S. Pat. No. 6,770,070 assigned to R. ITA Medical Systems, Inc. at col. 10, lines 14-17.) Cooling fluids may be provided to ensure the temperatures of energy transfer elements (on sealing and separating instruments) stay within a safe range. Cleaning solutions may be provided to ensure the surface of energy transfer elements stays free of materials such as loose tissue particles or charred tissue.

Step Four: Removal of the Minithoracoscope and Optional Insertion of CI Kink-Less, Non-Buckling Chest Tube, if Necessary

Once the internal inspection and sampling procedure is complete, a guide wire is introduced through the working miniport of the minithoracoscope and placed in a desired location. The CI minithoracoscope is then removed.

In many cases, once the CI minithoracoscope is removed, the procedure is complete and a chest tube need not be provided. For example, when the CI mininstruments used to obtain biopsy samples seal the site from which the sample is collected (prior to, simultaneously with, or shortly after separating the desired sample from the surrounding tissue), internal bleeding and drainage can be entirely avoided or at least substantially reduced. Use of the rapid tissue sealing and separating capabilities of modern technologies (including those that rely upon heat to both seal and separate) coupled with the small scale of the sampling instruments described herein has the advantage of avoiding the need for a chest tube in many cases.

Chest tubes are generally provided to compensate for incomplete sealing at the biopsy site during incision and sampling. Thus, a chest tube permits the drainage of blood, gases, and internal fluids over an extended period of time, as the biopsied site heals.

If a chest tube is found to be necessary, CI minidilators (trademark) are inserted first, along the tract the tube is to follow in order to enlarge the tract. A Seldinger technique can be used to position the chest tube. A single skin stitch can be used to secure the chest tube in position. Alternatively, other methods of securing the chest tube can be used if the stitch needs to be avoided.

Once the chest tube is properly in place within the interior of the patient, it is connected to a chest drainage system and 20 cm of suction is applied. A post-operative chest X-Ray should be obtained in the immediate post-operative period while the chest tube is in place.

Although any chest tube may be used with the methods of this invention, preferably the CI chest tube is used if a chest tube is determined to be necessary. The CI chest tube is highly desirable as compared with conventional chest tubes because, unlike most flexible chest tubes, it does not kink and does not buckle. Unlike most rigid chest tubes, the CI chest tube is not painful.

The CI chest tube comprises a long, hollow, tubular member with an outer core that is softer than the inner core. The softer outer core minimizes a patient's sensation of pain upon contact of the tube's external periphery with the surrounding bodily environment in which the tube is inserted. The more rigid structural integrity of the inner core minimizes the chance that the tube will buckle (blocking flow) upon bending as it is maneuvered internally. Within the walls of the tube's internal lumen is a deployable elastic element that can be activated from a proximal control site to remove kinks as they emerge, if they emerge. The internally deployable elastic element replaces the conventional trocar insertion method for removing tubular kinks.

Step Five: Removal of Optional Chest Tube

Chest tube removal is at the discretion of the pulmonologist. A band-aid may be applied after the chest tube is removed to protect the insertion area.

Next, several embodiments of the invention will be described as represented in the drawings.

An endoscopic system for lung biopsy comprises an instrument for sealing and separating a site to be biopsied. The sealing and separating instrument should be of a size capable of insertion through the working channels or miniports of a minithoracoscope. The instrument is designed to capture biopsy samples on the scale of 3 mm in diameter by 10 mm in depth. The small scale of the instrument is one feature responsible for its minimally invasive nature. However, in circumstances where more extensive invasion of the body can be tolerated, or where precaution and skill is used by the technician, the design of the present invention may be embodied in instruments of a larger scale.

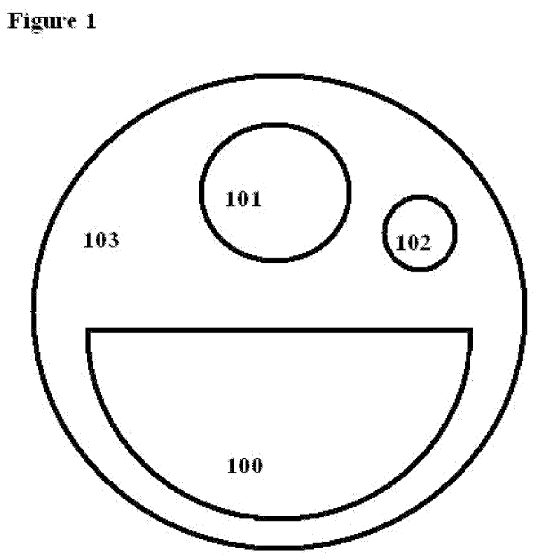

FIG. 1 shows a cross sectional view of a catheter 103 as part of an endoscopic system. The catheter has an atraumatic surface for protecting the body lining from agitation by the other instruments inserted therethrough. Alternatively, instead of a catheter, the other instruments and channels may be contained within a housing 103 of an endoscopic instrument system. The largest channel 100 should be reserved for the working instrument that will seal, resect (i.e. cut), and remove tissue samples. At least one other medium size channel 101 should be provided as a visualization port for insertion of a camera therethrough. Still other channels 102 can be provided for insufflation and/or drainage of fluids including gases and liquids

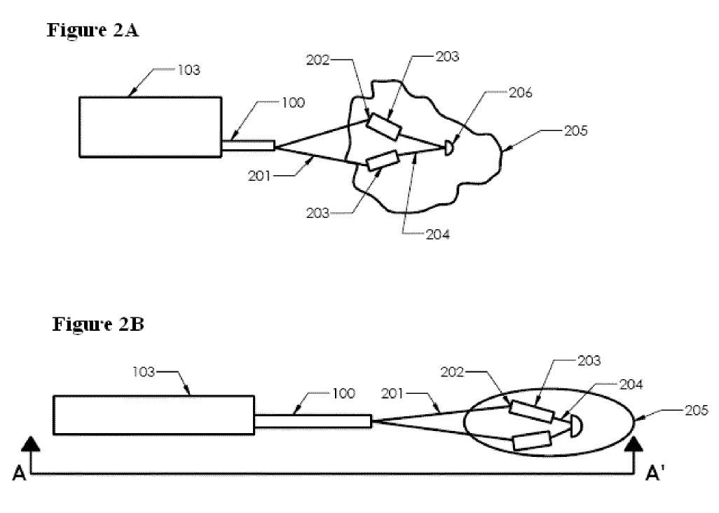

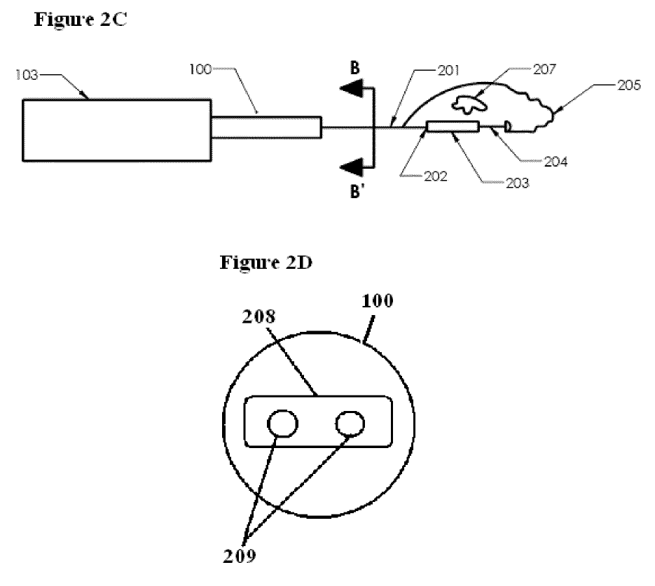

As illustrated in FIG. 2A-2B, a first embodiment 200 of an endoscopic system for lung biopsy comprises two (or more) long, stiff wires that extend distally from the end of a catheter 103 through a distal end of an instrument 100. This may be termed the “spring load wires” embodiment 200. The wires 201 initially extend outward, away from each other, as they exit the catheter 103. At a fixed pivot point 202 the wires 204 bend to extend inward, towards each other. The wires 204 converge completely to join together at their distal tips 206.

A kite or diamond shape is envisioned by the orientation of the wires 201, 204 in their extended (wire length), open (pivot point angle less than 180 degrees) position. However, a variety of other shapes may be formed at different times, depending upon the operator's control of the pivot points 202 and wires 201, 204 and depending upon the flexibility of the wires. The invention is neither dependent nor limited by the shape of the opening formed by the wire structure. Although the wires are generally stiff, the wire segments 201, 204 on each side of the hinge 202 need not be entirely straight but may curve or flex. The length of the outwardly extending segment of wire 201 on the proximal side of the pivot point 202 and the length of the inwardly extending segment of wire 204 on the distal side of the pivot point 202 need not be equal. The relative lengths of the wires 201, 204 on each side of the pivot point 202 will depend, in part, on how much wire 201 is extended from a distal tip of the instrument 100 through the spout of the catheter 103.

One or more sealing and separating element or anvil 203 is positioned on each wire 204 within the segment on the distal side of the pivot point 202. Each anvil 203 may be fixedly mounted within this wire segment 204 or slideable throughout the segment. An element capable of providing an energy source for sealing and separating a biopsy sample to be collected from its surrounding bodily environment is placed on at least one anvil. The other anvil(s) 203 need not have an energy source element thereon, but may instead serve as a base against which an anvil 203 possessing an energy source element can press to provide the pressure and/or energy intensity required to separate the bodily material held between the anvils 203. It is also possible that all anvils 203 have an energy source element thereon such that tissue gripped between two or more elements receives energy from more than one direction.

The size and dimensions of the anvils 203 and of the energy source elements thereon depend on balancing a number of factors. These factors include the size of biopsy sample desired and the dimensions of the catheter channel through which the sample must fit to be removed. For a minimally invasive procedure using an instrument inserted through the working channel of a minithoracoscope, anvils 203 of 3.5 mm length are desirable.

The two or more anvils 203 separate and come together as needed to grasp tissue at a site and then contract upon the tissue to apply the energy necessary to seal and/or separate. The opening and closing (expansion and contraction) of the anvils 203 depends upon the operation of the pivot points 202. The pivot points 202 of the wire structure can be operated by external controls which change their angle and cause them to bend, expanding the wire structure, or to straighten, contracting the wire structure. Another way to adjust the orientation of the pivot points 202 is to extend and retract the stiff wires 201 from the distal end of an instrument shaft 100 protruding through a catheter 103.

In addition to the anvils 203 (with sealing and separating elements thereon) positioned along the long, stiff wires 204, a collection bag 205 is also securely fastened to at least two wires. The collection bag 205 is designed to receive a biopsy sample 207 separated from the body by the anvils 203. The collection bag 205 is also designed to collect any material that passes through the kite or diamond-shaped structure of the open wires 201, 204 into the cavity created by the collection bag 205. Bending the pivot points 202 (from 180 degrees towards 90 degrees) causes the collection bag 205 to expand and its mouth-like entrance, formed by the kite-shaped wire structure, to open. Straightening the pivot points 202 causes the entrance of the collection bag 205 to close as the bag is sealed and expansion is terminated.

Once a biopsy sample 207 has been separated from its surrounding environment and the environment of the biopsy site has been sealed to prevent bleeding, the instrument and the sample are ready to be removed from the interior of the patient through the catheter channel 103. As illustrated in FIG. 2C, the pivot points 202 are completely straightened to align the anvils 203 and close the entrance to the collection bag 205 before the instrument is retracted from the body through the catheter channel 103.

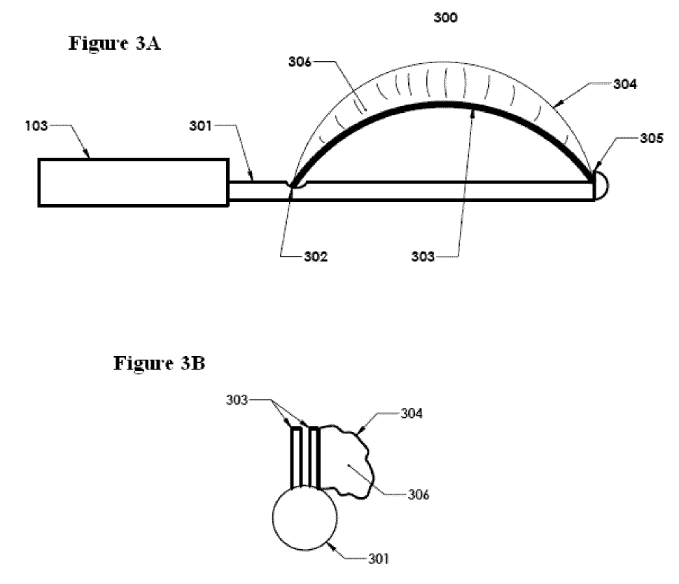

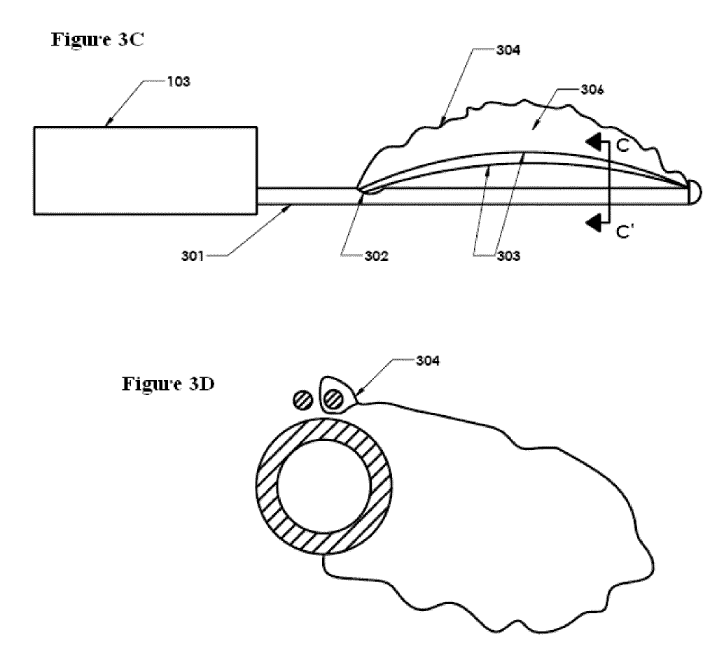

As illustrated in FIG. 3A-3D, an endoscopic system for lung biopsy according to a second embodiment 300 comprises one wire, two wires (as shown), or multiple wires wherein each wire 303 protrudes from an opening 302 within a rigid tubular mast 301 and is joined to the mast at its distal end 305. The mast 301 is extendible from the spout of a catheter 103. This may be termed the “mast and spinnaker” embodiment 300. Each wire 303 can be extended axially outward from the longitudinal direction of the rigid mast 301.

At least one of the wires 303 is capable of sealing and separating tissue to be biopsied by its connection to an energy source. The energy used to seal and separate may be provided uniformly along the entire length of the wire 303 (as shown) or confined to (or varied among) discrete elements (i.e. anvils) positioned along the length of the wire.

The sealing and separating wire 303 is extended from the tubular mast 301 in the shape of a rainbow until the area defined beneath its arch and the base of the mast is sufficiently large for the tissue to be biopsied to enter within it. Once the tissue to be sealed and/or separated is within the arch, the wire 303 is pulled taught against the mast 301 to apply pressure as the energy supplying mechanism is activated.

At least one wire 304 may be used to support a collection bag 306 for receiving biopsy samples that are separated from their surrounding environment. The collection bag support wire 304 need not have the capacity to seal and separate tissue as long as at least one other wire 303 is provided for this purpose. If the collection bag support wire 304 does not itself seal and separate tissue, then the wire 303 that does seal and separate should, preferably, be placed in close proximity and parallel to the bag support wire 304 in order to maximize the amount of sample collected. The support wire 304 should be extended from the tubular mast 301 to open the bag 306 for sample collection. Once a sample has been obtained, the support wire 304 can be retracted parallel to the mast 301 to close the bag 306 for sample removal.

The sample collection bag 306 may also be used independently to obtain some types of samples, rather than relying upon the sealing and separating wire 303 to cleave tissue before it enters the bag 306. For example, once the wire 304 supporting the sample collection bag 306 is extended, the mast 301 can be maneuvered in a sweeping motion to cause freely floating or loosely connected sample material (i.e. fluid, blood, cells, etc.) to enter the cavity created by the collection bag 306.

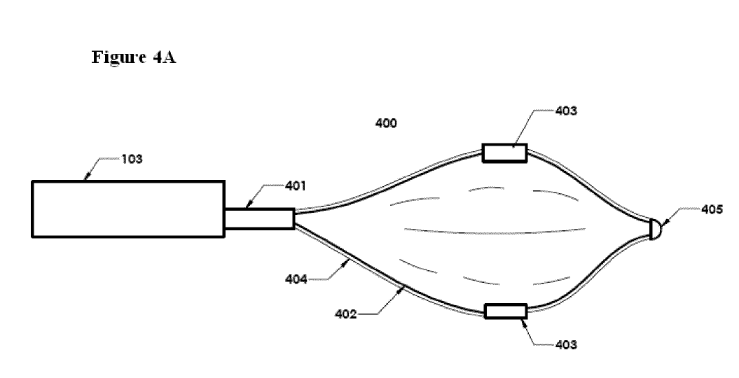

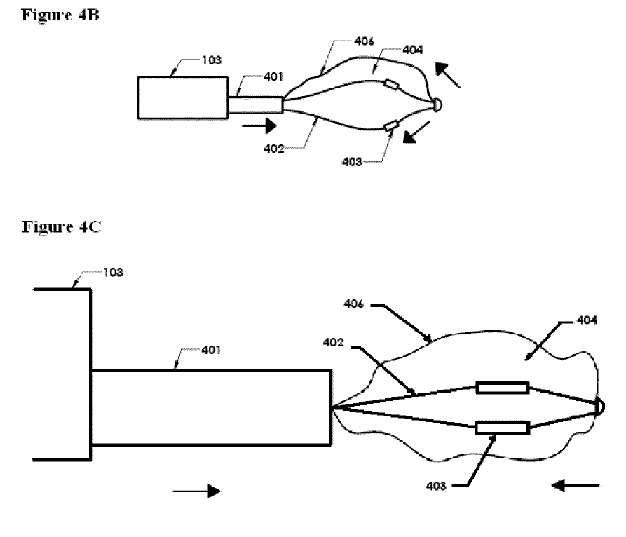

As illustrated in FIG. 4A-4C, an endoscopic system for lung biopsy according to a third embodiment 400 comprises two (or more) wires 402 extending from a distal end of an instrument 100 through a catheter (or endoscopic system housing) 103 and joined at their distal ends 405. This may be termed the “lasso” embodiment 400. The basic structure of this embodiment is similar to the structure of the first embodiment 200 shown in FIG. 2A-2C except that there is no pivot point 202. Thus, rather than forming a kite or diamond shape when opened, the structure formed by the wires of this embodiment lacks intermediate edges and resembles a radish or spinning-top in shape. The wires 402 used in this embodiment may be stiff (as in the first embodiment 200) or they may be flexible to accommodate and receive samples of varying shapes.

As in the first embodiment 200, the sealing and separating elements (or anvils) 403 on two or more adjacent wires 402 come together as the wires 402 are retracted from their proximal ends. Unlike the first embodiment 200, in which the anvils 203 were fixed on the distal side of a pivot point 202, the anvils 403 may be fixedly or slideably positioned anywhere along the length of the wires 402. If the position of the anvils 403 is slideable and not fixed, the position of the anvils 403 at the time of sealing and separation must be capable of being controlled because tissue to be sealed and/or separated must be grasped between two parallel opposing anvils 403. Therefore, a means for synchronizing the position of two or more anvils along the length of their wires 402 may be provided. The means for synchronizing the positions of two or more anvils 403 could include a magnetic system in which opposite magnetic charges are induced into two anvils 403 on different wires 402 causing them to be attracted towards one another. The means for synchronizing could also include a pulley system in which the position of the anvils 403 along their wires 402 is adjusted in incremental fixed units that can be regulated from a proximal control handle. A means for temporarily fixing the position of an anvil 403 along a wire 402 may also be provided. The means for temporarily fixing position could include a magnetic system in which a site on the wire 402 in which an anvil 403 is desired is induced with a magnetic charge and the anvil 403 is induced with an opposite magnetic charge to attract it to that site. The means for temporarily fixing position could also include a pulley system in which the position of an anvil 403 along its wire 402 can be adjusted in incremental fixed units subject to regulation from a proximal control handle. Therefore, although the position of the anvils 403 along their wires 402 may be made adjustable (i.e. by slideably mounting the anvils 403 along the wire 402), it may be controlled so that anvils 403 are not randomly freely sliding at all times.

As in the first embodiment 200, an element capable of providing energy for sealing and separating a biopsy sample to be collected from its surrounding bodily environment is placed on at least one anvil 403. An energy source (i.e. heating element) can be placed on more than one anvil 403, or on each anvil 403, but need not be. An anvil 403 without an energy source element can be used as a buttress against which another anvil 403 with an energy source element is held.

At least one collection bag 404 may be provided spanning the entire length of any two wires 402. As the wires 402 are extended and the anvils 403 slideably mounted thereon separate from one another, the entrance to the bag 404 situated between the wires 402 and anvils 403 opens for sample collection (see FIG. 4A and FIG. 4B). Withdrawing the wires 402 to draw opposing anvils 403 together closes the entrance to the collection bag 404 for securely removing the sample (see FIG. 4C).

Separate collection bag support wires 406 (without anvils) may also be provided as described for the second “mast and spinnaker” embodiment 300. However, in most cases attaching the collection bag 404 to the same wires 402 as those to which the anvils 403 are slideably mounted will maximize sample capture and retention. It is desirable that as much of the separated sample (i.e. tissue) as possible be collected within the bag 404 in order to obtain a sample of sufficient size for analysis with minimum effort and time. It is also desirable that as much of the separated sample (i.e. tissue) as possible be collected within the bag 404 in order to minimize the risk of freely floating separated material causing complications in other parts of the body.

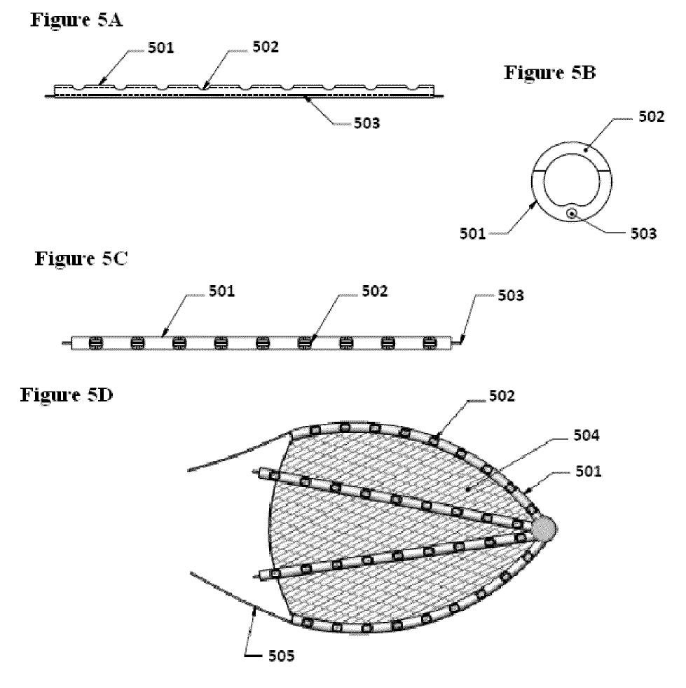

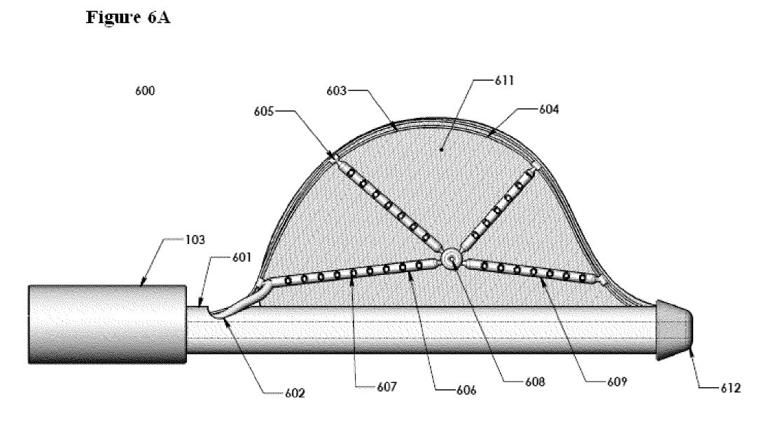

As illustrated in FIG. 5A-5D, an endoscopic system for lung biopsy according to a fourth embodiment 500 comprises one or more soft extruded tube 501, each with one or more opening 502 and optionally, one or more wire 503 therein, and through which vacuum suction may be applied. This may be termed the “octo-arm” (FIG. 5A-5C) 500 or “octo-pad”/“octo-mat” (FIG. 5D) embodiment 506.

Unlike the previous embodiments, which relied upon a collection bag 205, 306, 404 for sample removal, this embodiment does not require a collection bag 205, 306, 404. Instead a sample is removed by the application of vacuum suction through a soft extruded tube 501 in which the sample resides. The vacuum suction collection method may be better suited to sample shapes and sizes that are difficult to capture in a collection bag 205, 306, 404 with a high degree of efficiency. The vacuum method is also advantageous because it may be more likely to recover and remove very small bodily particles and debris set loose during the biopsy procedure. The removal of such debris, fluid, etc. by vacuum suction prevents them from remaining as residue to be drained with a chest tube post-surgery. Prompt removal of small debris, fluids, etc. by vacuum suction also prevents such materials from migrating to other parts of the body where they could potentially aggravate other systems before they can be removed with a drainage device.

To supplement the vacuum system in its capture and withdrawal of material, a mesh-netting network 504 may be provided between adjacent vacuum tubes (“octo-arms”) 501. As shown in FIG. 5D (an “octo-pad”/“octo-mat” embodiment 506 comprising more than one “octo-arm” 501), the netting 504 serves to cover gaps between adjacent vacuum tubes 501. The gap coverage provided by the netting 504 permits the retention of material missed by the tubes 501 and redirects it into the tubes 501 for subsequent attempts at being successfully suctioned into the interior of the tubes 501.

The most common order of operation for the seal, separate, and capture steps using this embodiment differs from the order followed in the other embodiments hereinbefore described. The first through third embodiments (200, 300, 400) were generally designed to seal a site to be biopsied, separate material from the site, and then capture the separated material. Alternatively, the first through third embodiments (200, 300, 400) may be used to separate tissue, seal the surrounding site, and then capture or to separate, capture, and then seal. Generally, capturing does not occur as the first step when using the first three embodiments (200, 300, 400) to obtain a biopsy sample. In contrast, this fourth embodiment 500 is designed to capture material prior to sealing and separation. Material at a site to be biopsied is first drawn into an opening of a soft extruded tube 501 by vacuum suction flowing through the tube 501. Next, energy generated by one or more wire 503 housed within the interior of the tube 501 (i.e. embedded within the internal perimeter of the tube) is used to seal and separate the captured material from its external bodily attachment.

To enhance the vacuum effect upon captured material residing in the distal end of the tube, the opening(s) 502 in the tube(s) through which material is captured may be made closeable. Once the capture, sealing, and separation of material are complete, the tubular opening(s) 502 may be closed to create a more intense vacuum that withdraws all material in the tube 501 through the proximal end of the tube and out through the catheter 103 for collection and analysis.

In the multi-tube “octo-pad”/“octo-mat” configuration 506 (see FIG. 5D) the perimeter of the tubes 501 may be connected via a drawstring 505. The drawstring 505 may be used to keep the tubes 501 together upon insertion and removal of the “octo-pad”/“octo-mat” 506 in order to reduce the total area occupied by the device. Optionally, the drawstring 505 may also be interconnected with the mesh-netting 504 between tubes 501, such that pulling the drawstring 505 tightly eliminates the mesh-netting areas 504 as adjacent tubes 501 move to abut each other.

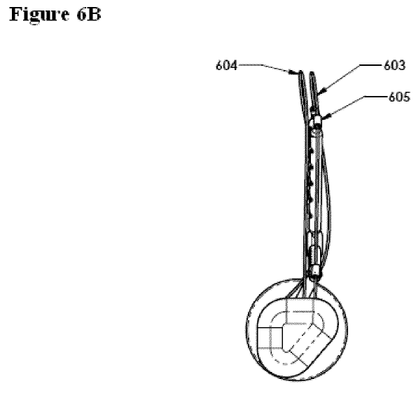

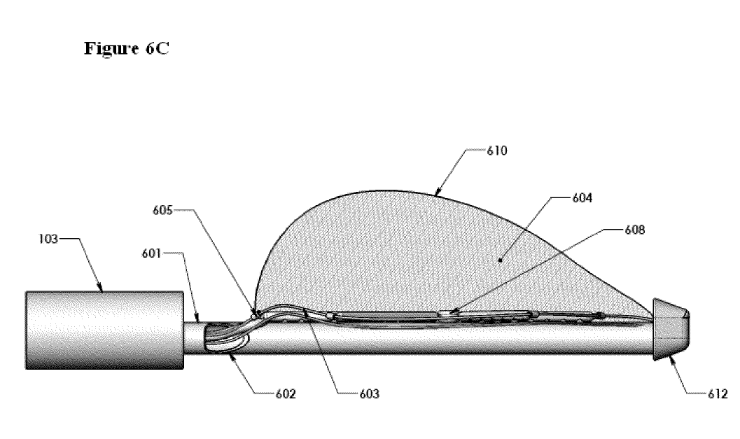

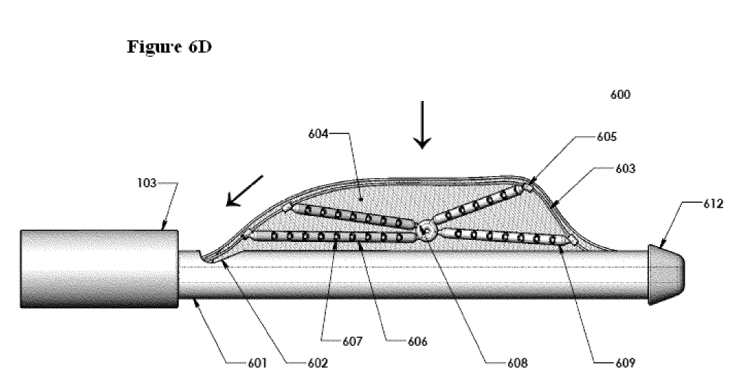

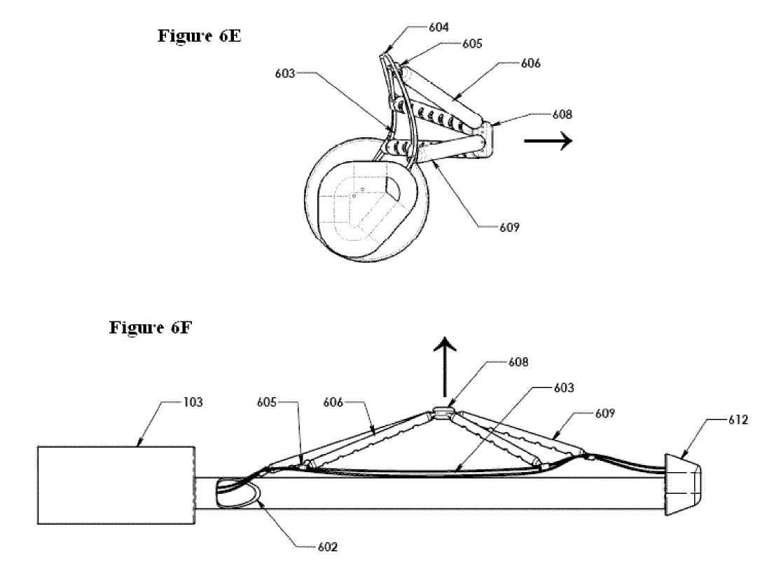

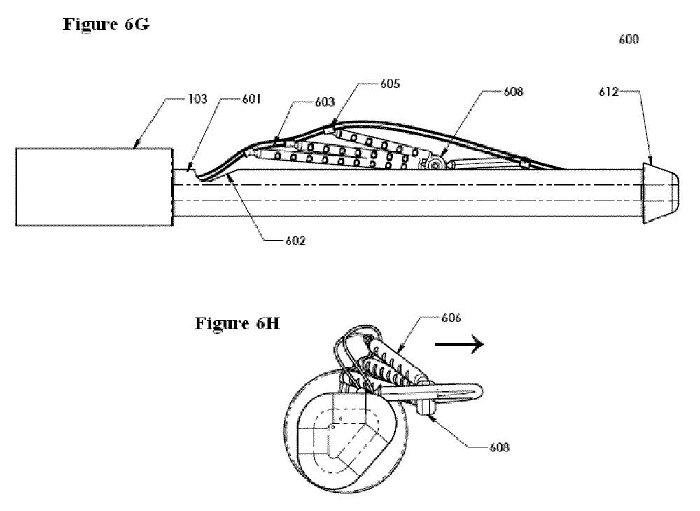

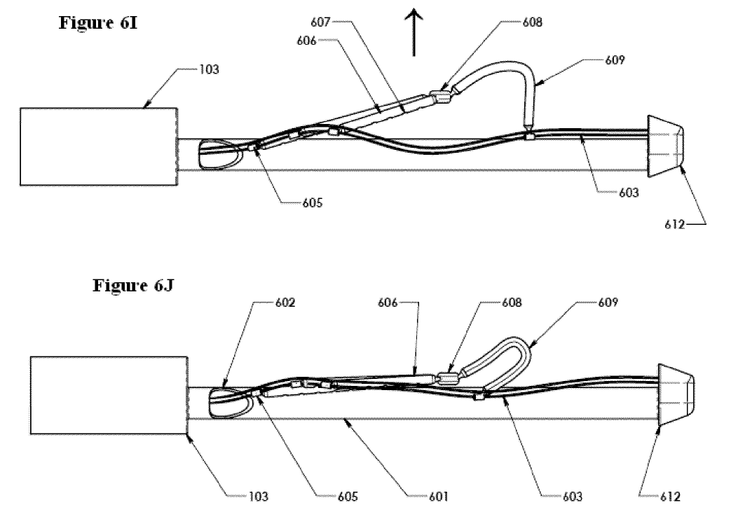

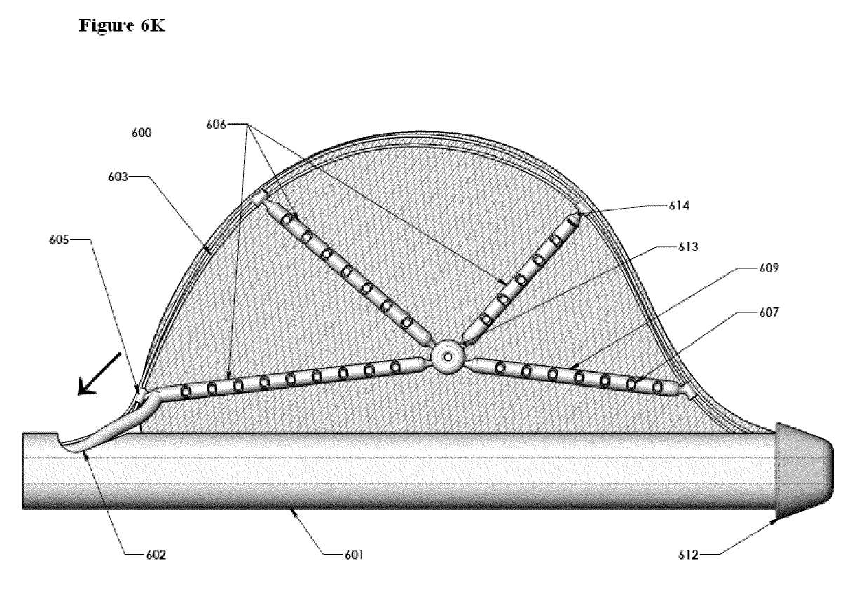

As illustrated in FIG. 6A-6K, an endoscopic system for lung biopsy according to a fifth embodiment 600 comprises one or more stiff extruded tube 606 and one or more flexible extruded tube 609 attached to a support wire 603 through flexible hinges 610, 611 and blocks 605 slideably mounted along the support wire 603. The support wire 603 extends from a hollow, tubular, rigid mast 601 similar to that in the “mast and spinnaker” design of the second embodiment 300. The difference is that in the second embodiment 300 the support wire 304 carried a collection bag 306 while in this embodiment 600 the support wire 603 carries several suctioning “octo-arms” 606, 609 as described in the fourth embodiment (501 in 500). Optionally, a collection bag 604 may also be provided in this embodiment and it may be carried by the same support wire 603 that carries the “octo-arms” 606, 609 or by a separate additional support wire 610. In addition to the support wire 603, a sealing and separating wire 611 also extends from an opening 602 in the proximal end of the mast 601 and is affixed at its other end to the distal end of the mast 612 to form the shape of a rainbow or arch in its extended position. This may be termed the “stiff mast with tissue grab and capture technology” embodiment 600.

The stiff extruded tubes 606 and flexible extruded tubes 609 are similar to the “octo-arms” 501 of the fourth embodiment 500. Each tube 606, 609 has at least one opening 607 through which vacuum suction may be applied.

As in the second embodiment 300, the entire length of the sealing and separating wire 611 may be used to uniformly transmit energy to the tissue to be severed. Alternatively, energy transmission may be confined to discrete elements (i.e. anvils) positioned along the length of the wire 611. Another alternative is to vary the degree or intensity of energy transmission (i.e. temperature level for heat energy) along the length of the wire 611 and/or between discrete energy transmission elements.

This fifth embodiment 600 is ideally suited for collecting a biopsy sample from a substantially planar surface such as the wall of a lung. First, the biopsy system is inserted through a catheter or a working channel 103 of an endoscopic instrument (i.e. a minithoracoscope) through a percutaneous sheath to enter the interior of the body in the region of interest. Insertion occurs with the system in its closed state in which tension is applied to all wires 603, 610, 611 to keep them drawn taught against the body of the mast 601 (see FIG. 6G). When the support wire 603 is fully withdrawn, the “octo-arms” 606, 609 initially extend radially perpendicular to the longitudinal direction of the mast. This conglomeration of “octo-arms” 606. 609 may then be rotated about the hinges 613, 614 of the arms to draw them alongside of the mast 601 (arms parallel to the longitudinal axis of the mast) so that the device takes up as little space as possible during insertion and withdrawal from the sampling site.

Following insertion of the system in its closed state, the system is deployed or opened. Extending the length of the support wire 603 from the mast 601 causes the blocks 605 along the wire, through which the “octo-arms” 606, 609 are attached, to spread out along the wire 603. As the blocks 605 spread out, the “octo-arms” 606, 609 also spread out until they all occupy the same plane beneath the arch created by the support wire 603. In its open, deployed state the biopsy system is moved into its sampling position against and parallel to a planar surface such as a lung wall.

Once it is ascertained that the biopsy system lies flat against a lung wall, vacuum pressure is applied to induce the suction ports 607 (openings on the tubes) to attach the tubes 606, 609 to the wall. Once the tubes 606, 609 have firmly attached themselves to the lung wall and are stably held in position by the maintenance of vacuum pressure, the operator can begin to withdraw the wires 603, 610, 611 (the sealing and separating wire 611, the “octo-arm” support wire 603, and any collection bag support wire 610).

As the wires 603, 610, 611 are pulled down toward the mast 601, the tubes 606, 609 deflect outward on their hinge points 613, 614 so that their boundaries define the periphery of a void space into which the lung sample is pulled. The sealing and separating wire 611 stays within the plane of the mast 601 as the wire 611 is pulled down. Tissue positioned between the mast 601 and the wire 611 is severed, causing it to enter the suctioning void space created by the configuration of “octo-arms” 606, 609.

The “octo-arms” 606, 609 collect the separated sample material in a manner similar to that described above with respect to the fourth embodiment 500. Sample material enters through the openings 607 of the tubes and is suctioned away from the body to a proximal collection site for analysis. It should be noted that a difference between this fifth embodiment 600 and the fourth embodiment 500 is that in the fourth embodiment 500, tissue is separated by a wire 503 within the tubes 501 after entering the tubes. In this embodiment, tissue is separated by a wire 611 outside the tubes 606, 609 before entering the tubes 606, 609. However, in an alternative design, the tubes 606, 609 of this embodiment or the mast 601 could also have internal wires (not shown) such that tissue could be sealed and separated both before and after entering the tubes 606, 609 and/or mast 601.

The present invention is not limited to the embodiments described above. Various changes and modifications can, of course, be made, without departing from the scope and spirit of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

The present invention is useful in the field of endoscopic systems that can grasp, seal, separate, and collect a biopsy sample and in the field of methods of obtaining biopsy samples.

What is claimed is:

1. An endoscopic system comprising:
a working channel;
an instrument within the working channel;
two or more wires extendable from the instrument;
wherein at least one wire is capable of transferring energy;
wherein the wires are joined at their distal ends.

2. The endoscopic system of claim 1, further comprising at least one anvil on each wire, wherein the wire(s) capable of transferring energy transfer(s) that energy through the anvil.

3. The endoscopic system of claim 2, further comprising a pivot point on each wire to permit angular movement.

4. The endoscopic system of claim 3, wherein each anvil is positioned on a segment of wire distal to the pivot point.

5. The endoscopic system of claim 2, wherein the anvils are slideable along the wire.

6. The endoscopic system of claim 2, further comprising a means for synchronizing movement of two or more anvils.

7. The endoscopic system of claim 2, wherein each anvil is 2-4 mm in length.

8. The endoscopic system of claim 1, further comprising a collection bag attached to any two wires.

9. An endoscopic system comprising:
a working channel;
an instrument within the working channel;
a stiff mast extendable from the instrument, wherein the mast has an opening therein;
one or more wires extendable from the mast at the opening, wherein the one or more wires are joined at distal ends to a distal end of the mast;
wherein at least one wire is capable of transferring energy to a sample held between the wire and the mast.

10. The endoscopic system of claim 9, further comprising a collection bag attached to at least one wire.

11. The endoscopic system of claim 10, wherein the collection bag is also attached to the mast or a second wire.

12. The endoscopic system of claim 9, further comprising one or more extruded tubes with one or more openings therein configured to conduct a vacuum pressure system for suctioning, wherein at least one extruded tube is connected to at least one wire.

13. The endoscopic system of claim 12, further comprising one or more block and one or more flexible hinge wherein at least one extruded tube is connected to at least one wire through at least one flexible hinge attached to at least one block mounted upon the wire.

14. The endoscopic system of claim 13, wherein at least one block is slideably mounted upon the wire.

15. The endoscopic system of claim 14, comprising two or more extruded tubes and further comprising a joint wherein all tubes are connected to each other through the joint.

16. The endoscopic system of claim 15, wherein at least one extruded tube is rigid and at least one extruded tube is flexible.

17. An endoscopic system comprising:
a working channel;
an instrument within the working channel;
one or more soft extruded tubes extendable from the instrument;
wherein each soft extruded tube has one or more openings therein configured to conduct a vacuum pressure system for providing suction.

18. The endoscopic system of claim 17, further comprising one or more wires within an interior of at least one soft extruded tube wherein the wires are configured to transfer energy to a sample held within the tube.

19. The endoscopic system of claim 18, comprising two or more soft extruded tubes, and further comprising a mesh-netting network and a drawstring wherein each tube is connected to at least one other tube by the mesh-netting network and the drawstring is attached to an outermost perimeter of the tubes.

20. A method for obtaining a biopsy sample from a body using the endoscopic system of claim 1, comprising:
insufflating a gas into an intrapleural region to collapse a lung to be biopsied;
inserting the instrument into a site from which a biopsy sample is desired;
extending two or more wires from the instrument to an open position for grasping the sample;
surrounding the sample with the wires;
retracting the wires to straighten them to a closed position with the sample held between wires;
activating a power source to provide energy to at least one energy transfer element on at least one wire;
transferring an amount of energy sufficient to seal from at least one energy transfer element on at least one wire to the sample;
continuing to transfer energy to the sample sufficient to resect from at least one energy transfer element on at least one wire to the sample;
collecting a separated sample with a collection bag or suction; and
removing a collected sample from the body by withdrawing the collection bag through the working channel or by suction.

21. A method for obtaining a biopsy sample from a body using the endoscopic system of claim 9, comprising:
insufflating a gas into an intrapleural region to collapse a lung to be biopsied;
inserting the instrument into a site from which a biopsy sample is desired;
extending the stiff mast from the instrument;
extending one or more wires from the opening of the mast to form an arch-shaped open position between the wire(s) and the mast for grasping the sample;
surrounding the sample with the arched wire and the mast;
withdrawing at least one wire within the mast so that the wire aligns against the mast in a closed position with the sample held between the wire and the mast;
activating a power source to provide energy to at least one wire;
transferring an amount of energy sufficient to seal from at least one wire to the sample;
continuing to transfer energy to the sample sufficient to resect from at least one wire to the sample;
collecting a separated sample with a collection bag or suction; and
removing a collected sample from the body by withdrawing the collection bag through the working channel or by suction.

22. A method for obtaining a biopsy sample from a body using the endoscopic system of claim 17, comprising:
insufflating a gas into an intrapleural region to collapse a lung to be biopsied;
inserting the instrument into a site from which a biopsy sample is desired;
extending one or more extruded tubes from the instrument;
suctioning the sample, directing it within one or more openings of one or more extruded tubes, by conducting vacuum pressure through one or more openings of one or more extruded tubes;
activating a power source to provide energy to one or more wires within one or more tubes;
transferring an amount of energy sufficient to seal from at least one wire to the sample;
continuing to transfer energy to the sample sufficient to resect from at least one wire to the sample; and
removing a collected sample from the body by conducting vacuum pressure through one or more tubes to direct the sample proximally and out through the working channel.
